(19) United States
(12) Patent Application Publication
(10) Pub. No.: US 2025/0204812 A1
(43) Pub. Date: Jun. 26, 2025

(54) SYSTEM AND METHOD FOR AUTOMATED PERITONEAL MEMBRANE FUNCTION ASSESSMENT SAMPLING METHODS FOR PD TREATMENT SYSTEMS

(71) Applicants: Fresenius Medical Care Holdings, Inc., Waltham, MA (US); Fresenius Medical Care Deutschland GmbH, Bad Homburg (DE)

(72) Inventors: David Yuds, Hudson, NH (US); Paul Chamney, Tring (GB); Eric Bergman, Newton, MA (US); Kerissa Adams, Norman, OK (US); Jun Yi, New York, NY (US)

(21) Appl. No.: 18/985,694

(22) Filed: Dec. 18, 2024

Related U.S. Application Data
(60) Provisional application No. 63/612,818, filed on Dec. 20, 2023.

(51) Int. Cl. A61B 5/145; A61M 1/28

(52) U.S. Cl. CPC A61B 5/14507; A61B 5/14532; A61M 1/282

(57) ABSTRACT

This disclosure provides methods and systems for peritoneal dialysis (PD) effluent sampling and measurement, which are compatible with all types of PD treatment, and would enable more frequent assessment so a patient's PD prescription can be updated routinely. The methods include withdrawing a plurality of samples of PD effluent from the patient's peritoneal cavity periodically during the dwell phase of a PD treatment cycle. The method further includes obtaining sensor measurements of the samples of PD effluent. The method may return each PD effluent sample to the patient's peritoneal cavity after the measurement. Alternatively, the PD effluent samples may be stored in a container without returning them to the patient's peritoneal cavity.

### CROSS-REFERENCE TO RELATED APPLICATIONS

This patent application claims the benefit of U.S. Provisional Patent Application No. 63/612,818, filed Dec. 20, 2023, which is incorporated by reference herein in its entirety.

### BACKGROUND

When renal function becomes impaired beyond a point where homeostasis can no longer be maintained, renal replacement therapy (RRT) is an essential life sustaining intervention for many patients. One form of RRT, suited for treatment of patients in the home setting is peritoneal dialysis (PD). In PD, the membranous lining of the patient's peritoneum acts as a natural semi-permeable membrane that allows the passage of water and solutes. At the start of a PD cycle, dialysis fluid is instilled in the peritoneal cavity, establishing an osmotic gradient between the dialysis fluid and the surrounding body tissues. This causes waste products, excess sodium and water to be drawn from body tissues into the peritoneal cavity. After a period of several hours, the osmotic gradient dissipates and the transfer of waste products and excess sodium in particular ceases. At this point, the spent dialysis fluid (PD effluent) is drained from the cavity denoting the end of the PD cycle. Usually, several cycles are required each day.

The amount of solute, water and sodium removed on a weekly basis by PD, is determined by the ‘dose’ variables of a PD prescription, the transport status of the peritoneal membrane, and the efficiency with which the peritoneal cavity can be drained of PD effluent.

As described above, PD depends on the movement of toxins and excess fluid across a patient's peritoneal membrane. Over time or due to infections and co-morbidities, the peritoneal membrane deteriorates. This membrane deterioration may affect transport characteristics, necessitating changes in PD inputs (such as number and duration of dwell sections and dialysate glucose strength) over time to most efficiently deliver optimal therapy.

Traditional methods for assessing membrane function, like the Peritoneal Equilibration Test (PET), are often employed at clinics to evaluate the transport characteristics of the peritoneal membrane in PD patients. PET yields valuable information, such as transport rates, which can assist patients in selecting an appropriate PD system. However, these conventional membrane assessment techniques are typically labor-intensive. For instance, completing a PET typically requires four hours of a nurse's time, involving multiple samples taken from the patient during several PD cycles. Such limitations result in infrequent utilization of these conventional membrane assessment methods.

As a result, patients new to PD are usually tested a few months into their therapy in order to tweak a generic prescription; afterwards, the PET may be performed only if there is a big change in a patient's ultrafiltration volumes. In other words, the therapy becomes “uncalibrated” over time and can lead to a less efficient therapy that can result in having to extend treatment times. This approach is not only costly and inefficient, but also places a heavy burden on patients.

### SUMMARY

This summary is provided to introduce a selection of concepts in a simplified form that are further described below. This summary is not intended to necessarily identify key features or essential features of the present disclosure. The present disclosure may include the following various aspects and embodiments.

In one example, a method of sampling of peritoneal dialysis (PD) effluent for a peritoneal membrane function assessment comprises: (a) fluidly connecting a PD effluent sampling system to a patient catheter, wherein the PD sampling system comprises: a coiled tubing line having a first end and a second end fluidly connectable to the patient catheter; and a sensor device configured to measure a glucose concentration of PD effluent flowing to and/or from the coiled tubing line through the patient catheter; (b) withdrawing, by uncoiling the coiled tubing line, a sample of PD effluent from a patient's peritoneal cavity through the patient catheter during a dwell phase of a PD treatment cycle; (c) measuring, using the sensor device, the glucose concentration of the sample of PD effluent; and returning, by recoiling the coiled tubing line, the sample of PD effluent to the patient's peritoneal cavity.

Alternatively or additionally to any of the examples above, the method can further perform steps (b)-(d) at least three times periodically during the dwell phase.

In some examples, the coiled tubing further comprises a user-actuated handle that is attached to the first end of the coiled tubing line. The user-actuated handle, based on user-input, uncoils and recoils the coiled tubing line.

In some instances, the PD effluent sampling system further comprises: a controller; a motor connectable to the first end of the coiled tubing line; and a power source operably connected to the controller and the motor, wherein the controller is configured to provide control instructions to the motor to coil or uncoil the coiled tubing line.

In some variations, the PD effluent sampling system further comprises: a shaft having a first end and a second end, wherein the first end of the shaft is connected to the motor, and wherein the second end of the shaft is attachable to the first end of the coiled tubing line, wherein the controller is further configured to provide control instructions to the motor to actuate the shaft to coil or uncoil the coiled tubing line.

In some examples, the second end of the shaft is coupled to a tubing connector. The first end of the coiled tubing line is attachable to the second end of the shaft through the tubing connector.

In some instances, the controller is further configured to: provide first control instructions to actuate the shaft to uncoil the coiled tubing line for a first amount of time to obtain a first sensor measurement from the sensor device; and provide second control instructions to actuate the shaft to recoil the coiled tubing line.

In some variations, the controller is further configured to: provide the first control instructions to actuate the shaft to uncoil the coiled tubing line for the first amount of time to obtain a second sensor measurement from the sensor device; and provide the second control instructions to actuate the shaft to recoil the coiled tubing line.

In some examples, the controller is further configured to: provide the first control instructions to actuate the shaft to uncoil the coiled tubing line for the first amount of time to obtain a third sensor measurement from the sensor device; and provide the second control instructions to actuate the shaft to recoil the coiled tubing line.

In some instances, the PD effluent sampling system further comprises a transmitter, and wherein the method further comprises: disconnecting the PD effluent sampling system from the patient catheter; and transmitting, by the PD effluent sampling system through the transmitter, the first, second, and third measurements to a connected health device.

In some variations, the PD effluent sampling system is connected between the patent catheter and a PD system tubing set through a T-connector.

In some examples, the method further comprises: measuring, using the sensor device, other properties of the sample of PD effluent. The other properties comprises at least one of: temperature; conductivity; and turbidity.

In some instances, the sensor device further comprises a turbidity sensor, and the turbidity sensor is configured to detect peritonitis based on the sample of PD effluent.

In some variations, the coiled tubing line is made of biocompatible and flexible material.

In another example, a method of sampling of peritoneal dialysis (PD) effluent for a peritoneal membrane function assessment comprises: (a) fluidly connecting a PD effluent sampling system to a patient catheter, wherein the PD effluent sampling system comprises: a fluid container having an expandable volume fluidly connectable to the patient catheter; and a sensor device configured to measure a glucose concentration of PD effluent flowing to and/or from the fluid container through the patient catheter; (b) withdrawing, by expanding the expandable volume of the fluid container, a sample of PD effluent from a patient's peritoneal cavity through the patient catheter during a dwell phase of a PD treatment cycle; (c) measuring, using the sensor device, the glucose concentration of the sample of PD effluent; and (d) returning, by decreasing the expanded volume of the fluid container, the sample of PD effluent to the patient's peritoneal cavity.

In another example, a peritoneal dialysis (PD) effluent sampling system comprises: a coiled tubing line having a first end and a second end fluidly connectable to a patient catheter; and a sensor device configured to measure a glucose concentration of PD effluent flowing to and/or from the coiled tubing line through the patient catheter. The coiled tubing line is configured to uncoil to withdraw a sample of PD effluent from a patient's peritoneal cavity through the patient catheter during a dwell phase of a PD treatment cycle. The sensor device is configured to measure the glucose concentration of the sample of PD effluent. The coiled tubing line is configured to recoil to return the sample of PD effluent to the patient's peritoneal cavity.

Alternatively or additionally to any of the examples above, the coiled tubing further comprises a user-actuated handle that is attached to the first end of the coiled tubing line, wherein the user-actuated handle, based on user-input, uncoils and recoils the coiled tubing line.

In some examples, the PD effluent sampling system further comprises: a controller; a motor connectable to the first end of the coiled tubing line; and a power source operably connected to the controller and the motor. The controller is configured to provide control instructions to the motor to coil or uncoil the coiled tubing line.

In some instances, the PD effluent sampling system further comprises: a shaft having a first end and a second end, wherein the first end of the shaft is connected to the motor, wherein the second end of the shaft is attachable to the first end of the coiled tubing line, and wherein the controller is further configured to provide control instructions to the motor to actuate the shaft to coil or uncoil the coiled tubing line.

In some variations, the second end of the shaft is coupled to a tubing connector. The first end of the coiled tubing line is attachable to the second end of the shaft through the tubing connector.

In some examples, the controller is further configured to periodically withdraw samples of PD effluent from the patient's peritoneal cavity during the dwell phase of the PD treatment cycle by: providing, for withdrawing each sample of the samples of PD effluent, first control instructions to actuate the shaft to uncoil the coiled tubing line for the first amount of time to withdraw the particular sample of the samples of PD effluent from the patient's peritoneal cavity; obtaining, from the sensor device, a glucose concentration measurement of the particular sample of PD effluent; and providing second control instructions to actuate the shaft to recoil the coiled tubing line to return the particular sample of the samples of PD effluent to the patient's peritoneal cavity.

In some instances, the system further comprises: a transmitter configured to transmit the glucose concentration measurements of the samples of PD effluent to a connected health device.

In some variations, the coiled tubing line is made of biocompatible and flexible material.

In some examples, the sensor device is further configured to measure at least one of: temperature; conductivity; and turbidity.

In some instances, the sensor device further comprises a turbidity sensor, and the turbidity sensor is configured to detect peritonitis based on the sample of PD effluent.

In another example, a method of sampling of peritoneal dialysis (PD) effluent for a peritoneal membrane function assessment comprises: (a) fluidly connecting a PD effluent sampling system to a patient catheter, wherein the PD effluent sampling system comprises: a tubing line configured to connect between a patient catheter and a PD tubing set; a sample container fluidly connected to the tubing line; a sensor device positioned along the tubing line and configured to measure a glucose concentration of PD effluent flowing from the patient catheter to the sample container; a first tube clamp mechanism configured to control flow from a patient's peritoneal cavity to the tubing line; and a second tube clamp mechanism configured to control flow from the tubing line to a drain; (b) withdrawing a sample of PD effluent from the patient's peritoneal cavity through the patient catheter during a dwell phase of a PD treatment cycle by opening the first tube clamp mechanism for a set duration enabling PD effluent to flow from the patient's peritoneal cavity to the sample container through the tubing line; (c) measuring, using the sensor device, the glucose concentration of the sample of PD effluent as it flows from the patient's peritoneal cavity to the sample container; and (d) closing the first tube clamp mechanism.

Alternatively or additionally to any of the examples above, the method can further perform steps (b)-(d) at least three times periodically during the dwell phase.

In some examples, the PD effluent sampling system further comprises a transmitter, and wherein the method further comprises: disconnecting the PD effluent sampling system by opening the first tube clamp mechanism and the second tube clamp mechanism; and transmitting, by the PD effluent sampling system through the transmitter, the at least three measurements to a connected health device.

In some instances, the PD effluent sampling system further comprises a controller, and the controller is configured to control individual clamp closures for the first tube clamp mechanism and the second tube clamp mechanism.

In some variations, the PD effluent sampling system further comprises a frangible positioned between the tubing line and the sample container. The method further comprises opening the frangible to allow a fluid flow to pass the tubing line into the sample container.

In some examples, the tubing line is integrated into the PD tubing set, and the PD tubing set is a gravity-assisted PD (CAPD) tubing set or an automatic PD (APD) tubing set.

In another example, a peritoneal dialysis (PD) effluent sampling system for a peritoneal membrane function assessment comprises: a tubing line configured to connect between a patient catheter and a PD tubing set; a sample container fluidly connected to the tubing line; a sensor device positioned along the tubing line and configured to measure a glucose concentration of PD effluent flowing from the patient catheter to the sample container; a first tube clamp mechanism configured to control flow from a patient's peritoneal cavity to the sample container through the tubing line; and a second tube clamp mechanism configured to control flow from the tubing line to a drain. The first tube clamp mechanism is configured to: open for a set duration to withdraw a sample of PD effluent from the patient's peritoneal cavity through the patient catheter during a dwell phase of a PD treatment cycle to allow the sensor device to measure a glucose concentration of the sample of PD effluent; and close after the set duration.

Alternatively or additionally to any of the examples above, the tubing line is integrated into the PD tubing set, and the PD tubing set is a gravity-assisted PD (CAPD) tubing set or an automatic PD (APD) tubing set.

In some examples, the system further comprises: a controller configured to control individual clamp closures of the first tube clamp mechanism and the second tube clamp mechanism.

In some instances, the controller is further configured to: (a) control the first tube clamp mechanism to open for the set duration to withdraw the sample of PD effluent from the patient's peritoneal cavity through the patient catheter; (b) cause the sensor device to measure the glucose concentration of the sample of PD effluent as it flows from the patient's peritoneal cavity to the sample container through the tubing line; and (c) control the first tube clamp mechanism to close.

In some variations, the controller is further configured to perform steps (a)-(c) for at least three times during the dwell phase.

In some examples, the system further comprises: a transmitter, wherein the controller is further configured to transmit the glucose concentration measurements of the samples of PD effluent to a connected health device.

In some instances, the system further comprises: a frangible positioned between the tubing line and the sample container, wherein the frangible when open allows a fluid flow to pass the tubing line into the sample container.

Further features and aspects are described in additional detail below with reference to the Figures.

### BRIEF DESCRIPTION OF THE DRAWINGS

FIG. 1 is a schematic diagram of an exemplary peritoneal dialysis machine, in accordance with embodiment(s) of the present disclosure.

FIG. 2A is a schematic diagram of an exemplary configuration of a fill phase of a peritoneal dialysis treatment, in accordance with embodiment(s) of the present disclosure.

FIG. 2B is a schematic diagram of an exemplary configuration of a dwell phase of a peritoneal dialysis treatment, in accordance with embodiment(s) of the present disclosure.

FIG. 2C is a schematic diagram of an exemplary configuration of a drain phase of a peritoneal dialysis treatment, in accordance with embodiment(s) of the present disclosure.

FIG. 3A is a schematic diagram depicting an exemplary implementation of a PD effluent sampling system, in accordance with embodiment(s) of the present disclosure.

FIG. 3B is a schematic diagram depicting an exemplary implementation of another PD effluent sampling system, in accordance with embodiment(s) of the present disclosure.

FIG. 4 is a schematic diagram depicting an exemplary implementation of another PD effluent sampling system, in accordance with embodiment(s) of the present disclosure.

FIG. 5 is a schematic diagram depicting an exemplary implementation of a PD tubing set, in accordance with embodiment(s) of the present disclosure.

FIG. 6 is a flowchart of an exemplary process for performing sample measurements using a PD effluent sampling system, in accordance with embodiment(s) of the present disclosure.

FIG. 7 is a flowchart of an exemplary process for performing sample measurements using a PD effluent sampling system, in accordance with embodiment(s) of the present disclosure.

### DETAILED DESCRIPTION

A PD treatment cycle, which is often referred to as an exchange, includes three phases-a fill phase, a dwell phase, and a drain phase. During the fill phase, fresh dialysate (PD fluid) is delivered to a patient's peritoneal cavity. Once delivered, the dwell phase begins. During the dwell phase, the PD fluid inside the peritoneal cavity absorbs waste and excess fluid from the patient's body as the peritoneum acts as a filter. The duration of the dwell phase (e.g., dwell time) may be set as part of a patient's PD prescription. Once the dwell phase is completed the drain phase can begin, during which the PD fluid/waste (e.g., PD effluent) can be drained from the patient's peritoneal cavity. Upon completion of the drain phase, a new PD treatment cycle (e.g., fill, dwell, and drain) may be initiated immediately or may be initiated later, depending on the type of PD being employed by the patient.

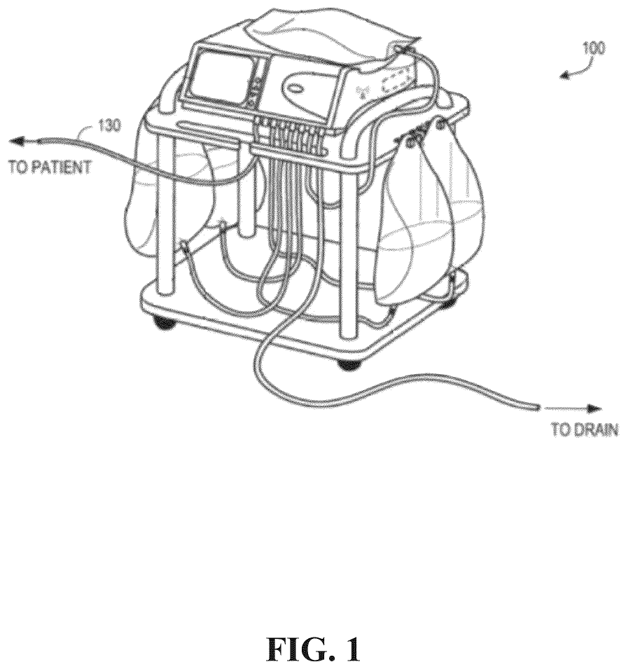

There are two primary types of PD, automated peritoneal dialysis (APD) and continuous ambulatory peritoneal dialysis (CAPD). APD is an automated process that uses a dialysis machine “cycler” to deliver (e.g., pump) the fresh dialysate to the peritoneal cavity during the fill phase and drain the PD effluent from the peritoneal cavity during the drain phase, and typically, the patient stays connected to the cycler throughout the course of a treatment cycle. Often, APD treatment is done while the patient is stationary (e.g., while sleeping). In APD, spent dialysate may drain into a bag, a sink, a toilet, or other drain location. FIG. 1 shows an example of a PD dialysis machine 100 configured to perform APD.

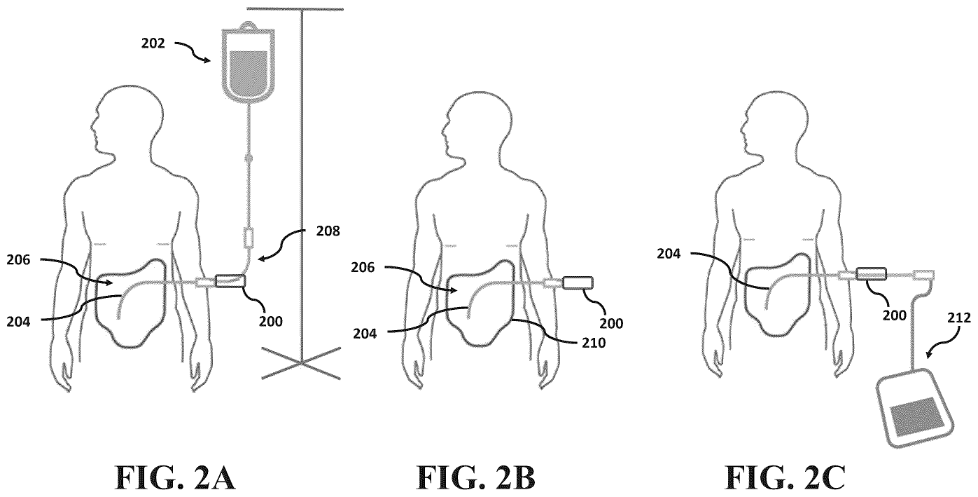

CAPD is a manual process and treatments are performed on an ongoing basis. With CAPD, for the fill phase, a patient connects a fresh dialysate bag to the patient's PD catheter (or sometimes referred to a patient line), and the fresh dialysate flows into the patient's peritoneal cavity via gravity. For example, FIG. 2A shows an exemplary configuration for a fill phase of a CAPD treatment. As shown in FIG. 2A, PD fluid 202 (e.g., fresh PD dialysate) may flow via gravity into a patient's peritoneal cavity 206 through a patient catheter 204 that may be surgically placed in the patient's peritoneal cavity 206. A PD tubing set 208 may be used to connect the PD fluid 202 to the patient catheter 204.

Once the fill phase is complete, the fresh dialysis bag can be disconnected, and the patient may move about during the dwell phase. For example, FIG. 2B shows an exemplary configuration for a dwell phase of a CAPD treatment. During the dwell phase, the PD fluid inside the peritoneal cavity absorbs waste and extra fluid from the patient's body as the peritoneum 210 acts as a filter. When the dwell phase is complete, the patient can perform the drain phase. FIG. 2C shows an exemplary configuration for a drain phase of a CAPD treatment. For the drain phase, the patient can connect the patient catheter 204 to a drain line, or a drain bag 212 and PD effluent can be drained from the peritoneal cavity to the drain line or the drain bag 212.

A PD prescription may include information including, but not limited to, PD fluid glucose concentration, PD fluid volume, dwell time for the patient, cycle frequency, and/or total number of cycles. The PD fluid glucose concentration is the amount of glucose concentration that is within the PD fluid. For example, PD fluid bags are available with various glucose concentrations (e.g., 1.5%, 2.3%, 4.25%). The PD fluid volume is the fill volume or the amount of liquid (e.g., the volume) that is administered to the patient for the PD treatment. For example, PD fluid bags of different volumes (e.g., 1.5 liters, 2 liters, 2.5 liters, 3 liters, 5 liters, or 6 liters) are available depending on the patient and PD dialysis type. The dwell time is the amount of time or duration that the dialysate stays within the patient (e.g., within the patient's peritoneal cavity).

Traditionally, a new PD patient starts out with a generic PD prescription, and then after a few months the patient undergoes a peritoneal membrane function assessment (e.g., a Peritoneal Equilibrium Test (PET)). The PET measures how well a patient's peritoneum transports waste and water. The following describes a commonly adopted standardized PET.

First, the patient drains out last night's PD effluent. Then, the patient lies down and fills the peritoneal cavity with fresh dialysate (e.g., two liters of 2.5% dextrose fluid) at 200 millileter (mL) per minute (min) (mL/min) for 10 minutes. Every two minutes, the patient rolls from side to side to be sure the fluid is well mixed in the patient's peritoneal cavity. As soon as all the fluid is in, the patient drains out 200 mL, and a 10 mL sample of PD effluent is taken out. The rest of the fluid is put back into the patient's peritoneal cavity.

Once this step is done, the patient walks or moves around for 2 hours while the fluid dwells. Then, a second sample of PD effluent is taken, and a blood sample is drawn. After 2 more hours of dwell time, the patient sits or stands to drain completely. This usually takes 20 minutes. The drain volume is checked, and another sample of PD effluent is taken. Each of the PD effluent samples and the patient's blood are tested to see how much glucose and creatinine (a waste removed by dialysis) are present. A PET can show if a patient's peritoneum has a high, average, or low transport rate. The test looks at the patient's drain volume, how much dextrose is left in the fluid sample, and how much creatinine is in the patient's drain fluid versus the patient's blood. Based on the results of the PET, a patient's initial generic PD prescription may be adjusted to provide for more effective treatment.

A patient's response to the PD treatments may and likely will change over time. For example, the patient's peritoneum membrane function may change over time. For instance, when a patient initially starts PD, the peritoneum may function efficiently. In other words, when dialysate is delivered to the patient, the peritoneum may help facilitate and/or act as a filter to remove waste product from blood. However, over time, the peritoneum functionality may deteriorate (e.g., based on morphological alteration of the peritoneum when it is used as a dialysis membrane), and might not operate as efficiently or effectively as the patient continues the PD treatment. Therefore, updating a patient's PD prescriptions overtime in response to these changes in order to provide more optimized treatment for the patient is beneficial. However, because the PET is labor intensive, it is usually performed infrequently. For example, after a patient undergoes an initial PET, a PET may not be performed again unless there is a big change in the patient's ultrafiltration volumes. This means that the patient's PD therapy becomes “uncalibrated” over time and can lead to a less efficient therapy that can result in having to extend treatment times. Eventually, the peritoneum functionality may lead to functional failure, and the patient may have to move onwards from PD treatment to another type of treatment such as hemodialysis treatment.

There is a need for a simpler, less labor-intensive, cost-effective method of membrane function assessment, that is compatible with all types of PD treatment, and that would enable more frequent assessment so the PD prescription can be updated routinely. Exemplary embodiments of the present disclosure provide systems and methods designed to address this need. As detailed herein, the present disclosure includes PD effluent sampling systems designed to perform a membrane function assessment by withdrawing one or more samples of PD fluid from a patient's peritoneal cavity and taking measurements of the samples using one or more sensor devices. The systems may be attached to the patient catheter or a line fluidly coupled to the patient line. The systems and methods described herein, may be suitable for use in the home environment, in a clinic or clinical environment, and/or in a hospital (e.g., critical care environment).

In some embodiments, the PD effluent sampling systems may incorporate appropriate components to withdraw a PD effluent sample for measurement and return the sample to the patient's peritoneal cavity after the measurement. This approach does not limit the number of samples by a total volume. In some embodiments, the PD effluent sampling systems may incorporate suitable components to withdraw PD effluent samples for measurement, without returning the samples to the patient's peritoneal cavity. As such, the overall volume of samples may be constrained by a predefined volume. The PD effluent sampling systems and methods described herein enable the retrieval and measurement of multiple PD effluent samples within a single PD treatment cycle. Furthermore, the PD effluent sampling systems and methods may be suitable for use by patients employing various types of PD (e.g., CAPD and/or APD).

Various exemplary embodiments of a PD effluent sampling system 200, according to the present disclosure, may be fluidly connected to the patient catheter 204, for example, as illustrated in FIGS. 2A, 2B, and 2C. During the dwell phase, the PD effluent sampling system 200 may be utilized to withdraw samples of PD effluent from the patient's peritoneal cavity 206 and perform measurements of the samples. For example, the PD effluent sampling system 200 may utilize one or more sensors to collect sensor measurements associated with the samples while the samples pass by the one or more sensors. During the dwell phase, which may last hours, the PD effluent sampling system 200 may periodically and/or intermittently retrieve a plurality of PD effluent samples (e.g., three or more samples) from the patient's peritoneal cavity, and obtain sensor measurements for each of the samples.

As detailed below, the PD effluent sampling system 200 may be configured in a variety of configurations and flexibly connect to patient catheters and PD tubing sets and/or PD systems with appropriate peripheral settings. For example, PD effluent sampling system 200 may be connected to a patient catheter during the fill phase and dwell phase. In some implementations, PD effluent sampling system 200 may be connected during the fill phase and dwell phase, and then may be disconnected at the end of the dwell phase or remain connected during the drain phase. This can enable calibration of the sensors of the PD effluent sampling system 200, to be done on fresh PD dialysate as it is supplied to the patient's peritoneal cavity during the fill phase. In some instances, sensor measurements of the PD effluent may be obtained during the drain phase as well.

In some examples, the PD effluent sampling system 200 may be configured to perform measurements exclusively during the dwell phase. As such, the system may be selectively connected to a patient line specifically during the dwell phase. While this approach may introduce some complexity for users, requiring them to disconnect and reconnect various tubing lines, it may enable cost savings by avoiding the use of more intricate connectors that enable connection during the fill phase and/or drain phase.

The PD effluent sampling system 200 may be utilized for one or more types of peritoneal dialysis system, including APD and/or CAPD. For either type, the PD effluent sampling system 200 may be connected to the patient catheter, at least during the dwell phase, and configured to withdraw samples and perform measurements during the dwell phase of a treatment cycle. For example, for CAPD, PD effluent sampling system 200 may be connected as shown in FIGS. 2A, 2B, and/or 2C.

For APD, a patient may set up a dialysis system (e.g., the dialysis machine shown in FIG. 1), and the dialysis system may perform the dialysis treatment automatically. In other words, for APD, a dialysis machine may be used to deliver and drain the dialysate automatically, with minimal human intervention. For APD, the PD effluent sampling system 200 may be connected, for example, inline between line 130 and a patient catheter (not shown in FIG. 1).

In some implementations, a user device (e.g., a personal computer, a smartphone, or other suitable computing entity) may be configured to receive sensor measurements from the PD effluent sampling system 200 during the dwell phase or at some time after the dwell phase of the respective treatment cycle ends. The user device and/or another computing entity may use the sensor measurements to calculate membrane transfer parameters, which may be evaluated and utilized to update a patient's PD prescription. The user device may further provide alerts to the patient such as when the dwell time has been reached and/or when the patient should take the sensor measurements.

In some implementations, a hybrid approach between the CAPD and the APD may be adopted for one or more treatment cycles. It will be appreciated that the components and/or operations for PD treatment described herein are merely exemplary, and that the principles discussed herein may also be applicable to other situations or examples.

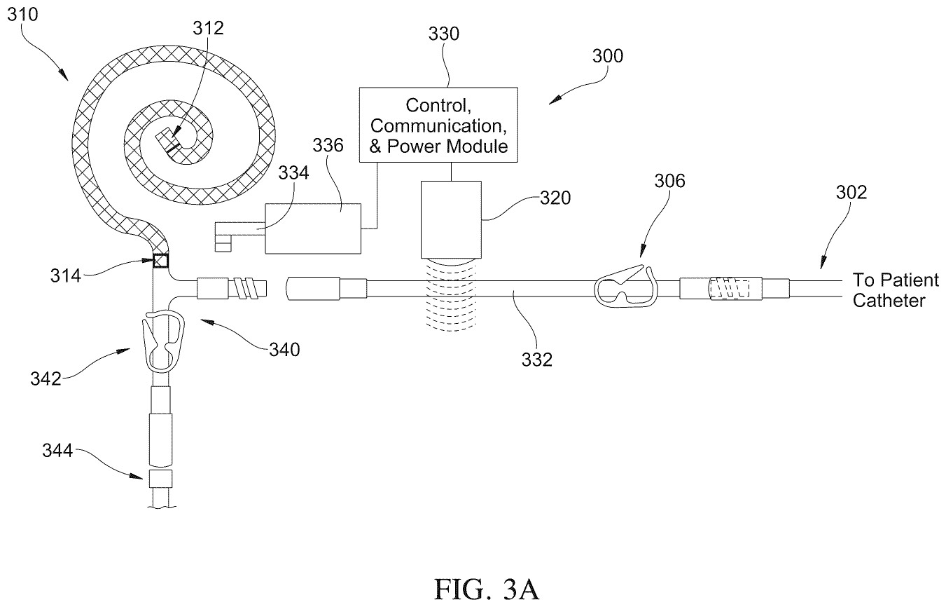

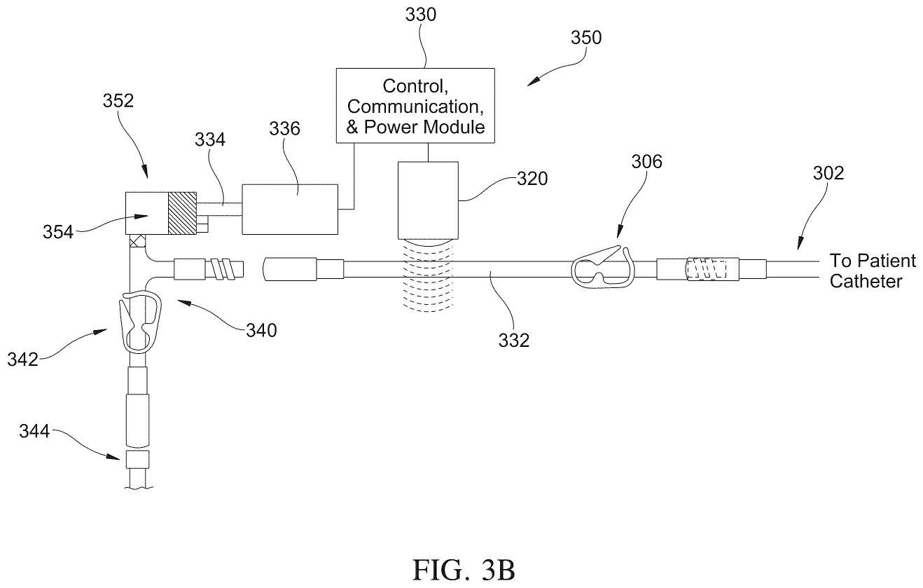

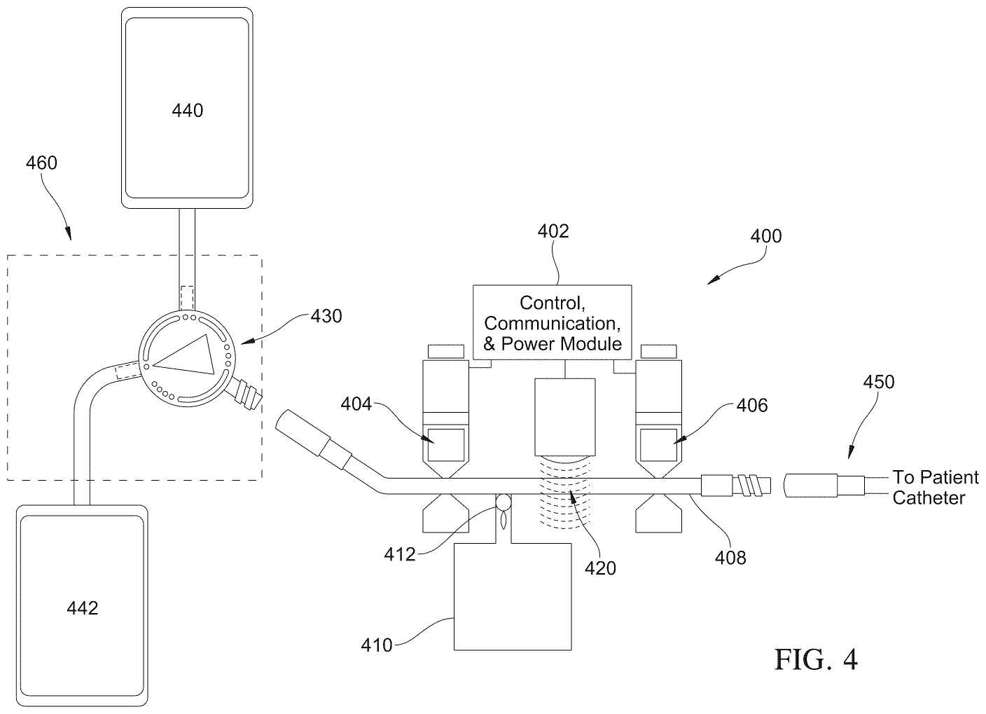

The present disclosure includes a number of exemplary embodiments of PD effluent sampling system 200. For example, FIGS. 3A, 3B, and 4 depict different embodiments of PD effluent sampling systems suitable for use as PD effluent sampling system 200.

FIG. 3A is a schematic diagram depicting an exemplary embodiment of a PD effluent sampling system 300 according to one or more examples of the present disclosure. In this example, a tri-end connector is utilized, enabling the PD effluent sampling system 300 to connect to a patient line 302 during a fill phase. It will be appreciated that other appropriate connections may be utilized to allow a PD effluent sampling system to connect to a patient catheter at different stages (e.g., during the fill phase or the dwell phase).

Referring to FIG. 3A, the PD effluent sampling system 300 may include a coiled tubing line 310 configured to withdraw/return one or more samples of PD effluent. The PD effluent sampling system 300 may also include one or more sensors (e.g., a glucose sensor 320) configured to perform measurements on the samples of PD effluent passing by the one or more sensors. In this example, the coiled tubing line 310 has a first end 312 and a second end 314 configured to fluidly connected a T-connector 340. The T-connector 340 can be connected to an end of the patient catheter 302 or a catheter tubing extension 332 (e.g., as shown in FIG. 3A). As shown in FIG. 3A, the glucose sensor 320 may be coupled, positioned against, or proximate to the tubing extension 332. In other implementations, the glucose sensor 320 may be coupled, positioned against, or proximate to the patient catheter 302, for example, if a tubing extension 332 is not being utilized.

In some variations, the sensor 320 may represent a plurality of sensors, either integrated in a single module or provided as separate components. The plurality of sensors may include various types of sensors, including a temperature sensor, a conductivity sensor, a turbidity sensor, or any combination thereof. A turbidity sensor may be configured to detect peritonitis based on the turbidity of a PD effluent sample.

Before use, the coiled tubing line 310 may include, for example flat tubing that may be made of any biocompatible and flexible material. In some examples, before use, the first end 312 of the coiled tubing line 310 may be wound tightly with the tubing pre-wrapped in a spool or insertable. A coiling action restricts fluid from flowing into the length of the tubing line 310. Conversely, an uncoiling action causes fluid to flow into the tubing line 310. The coiling and uncoiling actions may be motor driven or performed manually by a user with a crank such as a capstan or user-actuated handle attached to the first end 312. In some implementations, a clamp 306 may be positioned on tubing extension 332 and a clamp 342 may be positioned on the T-connector 340, as shown in FIG. 3A. During a dwell phase, the clamp 306 may be left open and the clamp 342 may be closed. As such, the coiled tubing line 310 may be operated to cause PD effluent to flow into and out of the tubing line 310, thereby causing the sample of PD effluent to flow by the glucose sensor 320.

During operation, the coil tubing line 310 may be contained in a housing to prevent obstructions and interference. The coiled tubing line 310 may not have to be fully uncoiled, just loosened for the volume to be filled with a suitable amount for sampling.

In further examples, the PD effluent sampling system 300 may include a controller 330 and a motor 336 configured to rotate a shaft 334. The PD effluent sampling system 300 may further include a power source that is operably connected to the controller 330, the motor 336, and a communication device (e.g., a transmitter) to transmit measurement data to another device.

The shaft 334 may include two ends. A first end of the shaft 334 may be connected to the motor 336, and the second end of the shaft 334 may be attachable to the first end 312 of the coiled tubing line 310. In some instances, the second end of the shaft 334 may be coupled to a tubing connector. The first end 312 of the coiled tubing line 310 may be attachable to the second end of the shaft 334 through the tubing connector.

The controller 330 may be configured to control the motor 336 and thereby control the coiling and uncoiling of the coiled tubing line 310. In some variations, the controller 330 may be programmed with instructions to energize the motor 336 and rotate the shaft 334 to uncoil the coiled tubing line 310 for a first amount of time to obtain a first sensor measurement from the glucose sensor 320. Additionally, and/or alternatively, the controller 330 may be programmed with instructions to energize the motor 336 and rotate the shaft 334 in the opposite direction to recoil the coiled tubing line 310 thereby causing the withdrawn PD effluent samples to be returned to the patient's peritoneal cavity.

In further examples, through the T-connector 340, the PD effluent sampling system 300 may be fluidly connected to a PD tubing set 344 (e.g., CAPD or APD tubing set) and the clamp 342 may be utilized to control (e.g., allow or stop) the flow to the PD tubing set 344. In some implementations, the PD effluent sampling system 300 may be connected to the patient catheter during the fill phase, during which, the coiled tubing line 310 may be wound tightly to prevent PD fluid flowing into the tubing line 310 as it flows through T-connector 340 to patient catheter 302. Alternatively, other types of connectors (or extension lines), such as standard tubing lines with two ends, may be utilized to connect the coiled tubing line 310 to the patient line 302. In this setup, the coiled tubing line 310 may be disconnected during the fill phase and subsequently connected during the dwell phase to withdraw samples and perform measurements.

In operation, the PD effluent sampling system 300 may perform sampling and measurement at least three times periodically during a dwell phase. For example, at specific time intervals, such as the zero-hour, two-hour, and four-hour marks (or other periods preferred by the user or desired for sampling by a care provider), the motor 336 may unwind the coiled tubing line 310 to draw a PD effluent sample from the patient for testing as it passes the glucose sensor 320 and filling the expanding volume of coiled tubing line 310. The glucose sensor 320 may measure the PD effluent sample. The PD effluent sampling system 300 may wirelessly transmit the data to another device, such as a smartphone, a dialysis machine, or any other suitable type of connected health device. After each draw, the motor 336 may recoil the coiled tubing line 310 to push the PD effluent back to the patient's peritoneal cavity so there is no change in volume or concentration caused by sample measurements. In some instances, the PD effluent sampling system 300 may be disconnected from the patient when the dwell phase ends and/or the measurements have been completed.

In some implementations, the first end 312 of the coiled tubing line 310 may be coupled with a user-actuated handle. A user (e.g., the patient or a caregiver) may uncoil and recoil the coiled tubing line 310 by operating (e.g., rotating) the user-actuated handle.

In some implementations, the PD sampling system 300 may come as a kit and some of the components may be designed to be durable (reusable) while other components may be designed to be disposable. For example, one or more coiled tubing lines 310, T-connectors 340, and corresponding clamps 342 may come in a kit and these may be disposable components. In some implementations, the kit may also include one or more tubing extensions 332 and corresponding clamps 306, which may also be disposable components. The durable components of the kit may include a glucose sensor 320 and a controller 330. The kit may further include additional components depending on whether it is a manual version or an automated version of the PD effluent sampling system 300. For example, the manual version may further include a user-actuated handle configured to attach to first end 312 enabling a user to uncoil and recoil the coiled tubing line 310. The user-actuated handle could be a disposable component or a durable component. The automated version of the kit may further include a motor 336 and a shaft 334, which may be operably connected to the controller 330. The motor 336 and shaft 334 may be durable components.

The following describes the steps a user (e.g., a patient and/or caregiver) may perform as part of an exemplary method of performing a PET by utilizing a PD effluent sampling system 300 with a CAPD setup. First, a patient may aseptically connect the T-connector 340 to the tubing extension 332 (or patient catheter 302 if no tubing extension 332 is being utilized) and then to the PD tubing set 344. Conversely, if the patient were not performing a PET, typically the patient would connect the patient catheter 302 to the PD tubing set 344 either directly or with tubing extension 332. In some implementations, the coiled tubing line 310 may come preconnected to the T-connector 340, in other implementations the patient may connect the coiled tubing line 310 to the T-connector 340. Next, the patient can connect the durable components (e.g., the glucose sensor 320, shaft 334, motor 336) of the PD effluent sampling system 300 to the coiled tubing line 310 and the tubing extension 332. The coiled tubing line 310 may be held in place (i.e., coiled), to prevent it from filling in the next step. Next, the patient can open the clamp 306 to initiate draining of PD effluent from the patient's peritoneal cavity to a drain line or drain bag (not shown in FIG. 3A) connected via the PD tubing set 344. After draining, the patient may initiate a flush of the tubing. The patient may then close the fluid pathway to the PD tubing set 344, for example by closing the claim 342. Next, the patient may perform a fill phase by connecting a fresh dialysate supply (e.g., a PD fluid bag) via the PD tubing set 344 and opening the claim 342. In some embodiments, an initial calibration of the glucose sensor 320 may be performed during the fill phase while fresh dialysate passes by the glucose sensor 320 as it flows through the tubing extension 332. Alternatively, in some embodiments, calibration may be performed at the start of the dwell phase. Once the fill phase is complete, the patient may close the clamp 342, disconnect the dialysate supply, and keep the clamp 306 open.

Next, the dwell phase may begin and the PET may proceed. In some embodiments, at the start of the PET process, an initial sample of PD effluent may be withdrawn from the peritoneal cavity for calibration purposes if calibration was not done during the fill phase. The sample of PD effluent may be withdrawn by uncoiling the coiled tubing 310. This action may be initiated by the patient, for example, by either manually uncoiling the coiled tubing line 310 (e.g., using the user-actuated handle) or it may be electronically controlled by buttons on the controller 330 (e.g., a display not shown in FIG. 3A) provided by the PD effluent sampling system 300) or a patient's linked smart device that communicates with the controller 330. The uncoiling of the coiled tubing line 310 causes PD effluent to be withdrawn from the peritoneal cavity via the patient catheter 302, through the tubing extension 332, and passed the glucose sensor 320. Once a desired volume of PD effluent is withdrawn, the coiled tubing line 310 may then be re-coiled to push the sample back to the patient's peritoneal cavity. Re-coiling could be manual or electronically controlled, depending on the version of the PD effluent sampling system 300 (e.g., manual or motorized/automated version). This initial sampling of PD effluent for calibration can be used to initiate a timer marking the beginning of the dwell phase of the exchange. If calibration was performed during the fill phase, this initial sampling may be skipped.

Periodically during the dwell phase, samples of PD effluent may be withdrawn and measured. The timing for this action may be set and triggered based on a timer. At the first measurement interval time mark, the coiled tubing line 310 may be uncoiled for a specified amount of time to withdraw a volume of PD effluent from the patient's peritoneal cavity through the tubing extension 332 and pass it by the glucose sensor 320, enabling measurement of the sample. After the sample has been measured, the coiled tubing line 310 may be recoiled, pushing the PD effluent sample back into the patient's peritoneal cavity. These steps for withdrawing a sample and getting a measurement of the sample may be repeated at a second measurement interval time mark, a third measurement interval time mark, and additional measurement time marks, depending on the instructions of the PET or the desired number of sample measurements.

Upon completion of the PET and the end of the dwell, the patient may connect the T-connector 340 to a drain line or drain bag via the PD tubing set 344, and initiate a drain by opening the clamp 342. Once the drain is completed, the clamp 306 can be closed and the sampling system 300 (e.g., glucose sensor 320, controller 330, and motor 334) may be removed. The sampling system 300 may transmit/broadcast the glucose sensor's measurements to a connected health device following the completion of the PET or during the PET process, for example, following the completion of each measurement. Subsequently, after the patient has drained, flushed the tubing, and filled their peritoneal cavity with fresh PD dialysate (if another exchange is to be performed), they may disconnect the T-connector 340 from the tubing extension 332 once the clamp 306 is closed. If a subsequent exchange is not being performed, the patient can remove the T-connector 340 following the drain and flushing.

FIG. 3B is a schematic diagram depicting another exemplary embodiment of a PD effluent sampling system 350 according to one or more examples of the present disclosure. As depicted in FIG. 3B, most of the components of the sampling system 350 may be the same as the sampling system 300 in FIG. 3A. The primary difference for the sampling system 350 is that, rather than a coiled tubing line 310, a fluid container 352 having an expandable volume 354 may be utilized. The fluid container 352 may adopt various forms, such as a bellow chamber or a syringe type container. The fluid container 352 may be contracted or expanded through a movable component within the fluid container 352. Decreasing the expandable volume 354 of the container 352 expels fluid from the container, while expanding the expandable volume 354 draws fluid into the container.

Similar to the setup illustrated in FIG. 3A, the moving component of the fluid container 352 may be actuated by a motor 336 via, for example, a shaft 334 or other moveable driver (e.g., piston or actuator). As such, the motor 336 may cause the expandable volume 354 of the fluid container 352 to increase or decrease based on movement of the shaft 334. In some instances, the controller 330 may include instructions based on user-input or predefined programs, and subsequently control the motor 336 to control movement of the shaft 334. In some variations, the moving part of the fluid container 352 may have a handle that allows a user to manually contract/expand the fluid container 352.

FIG. 4 is a schematic diagram depicting another exemplary implementation of a PD effluent sampling system 400 according to one or more examples of the present disclosure. As shown in FIG. 4, the PD effluent sampling system 400 may include a sample container 410 (e.g., an aliquot sample container), a sensor 420, a controller 402, a tubing line 408, and two tube clamp mechanisms 404/406. The tubing line 408 may be arranged to establish a connection between a patient catheter 450 and a PD tubing set 460 (e.g., a CAPD tubing set as shown in FIG. 4) or a connector switch 430, which may be separate from or a part of the PD tubing set 460. The sample container 410 may be connected to the tubing line 408. For instance, a length of tubing (e.g., the tubing line 408) may contain a branch to the sample container 410 and fit into a durable device consisting of the tube clamp 406/408, the sensor 420, and the controller 402, which may include a power supply, a communication module (e.g., a transmitter) to connect to another device (e.g., a smartphone, a tablet, a computer, a dialysis machine, etc.), and some indicators (e.g., through the controller 402) to indicate when flow is initiated and the (automatic or manual) membrane functional assessment begins.

In some examples, a frangible 412 may be positioned at the point where the tubing line 408 connects with the sample container 410. Breaking the frangible 412, which may be performed by the PD effluent sample system 400 or the patient, can allow fluid to flow into the sample container 410 from the tubing line 408. In some other embodiments, rather than the frangible 412, the PD effluent sampling system 400 may include an additional clamp mechanism at the same location as the frangible, which may be controlled (open/closed) by the controller 402.

The sensor 420 may be positioned along the tubing line 408 and configured to measure one or more parameters (such as a glucose concentration of PD effluent) of the PD effluent flowing from the patient catheter 450 to the sample container 410. In some variations, the sensor 420 may represent a plurality of sensors, either integrated in a single module or provided as separate components. The plurality of sensors may include various types of sensors, including a temperature sensor, a conductivity sensor, a turbidity sensor, or any combination thereof. A turbidity sensor may be configured to detect turbidity which may be used to detect peritonitis based on a sample of PD effluent.

The tube clamp mechanisms 404 and 406 may be positioned on opposite sides of the tubing line 408, respectively. The tube clamp mechanism 406 may control flow from the patient catheter 450 to the tubing line 408. The tube clamp mechanism 404 may control flow from the tubing line 408 to the PD tubing set 460 and/or the connector switch 430. For instance, closing a tube clamp mechanism prevents flow through a corresponding portion of a tubing line, while opening a tube clamp mechanism allows flow through the corresponding portion of a tubing line. The tube clamp mechanisms may be controlled by the controller 402, as shown in this example. Additionally and/or alternatively, the tube clamp mechanisms may be manually operated.

The controller 402 may be operably connected to the sensor 420, the tube clamp mechanisms 404/406, and other suitable components (e.g., transmitters, receivers, etc.). A power source may be connected to these components to supply power. The controller 402 may be programmed with instructions to control one or more components in the PD effluent sampling system 400 to control the overall operation. For example, the controller 402 may control the sensor 420 to take measurements, control the tube clamp mechanisms 404/406 to control individual clamp closures thereof, and/or control a transmitter to control transmission of the collected sensor measurements. Furthermore, the controller 402 may monitor and provide indicators for the operational status of the components connected to it. The indicators may include LED lights, a digital screen displaying text/graphic symbols, microphones sending audible alerts, and more.

In this example, one end of the tubing line 408 may be connected to the PD tubing set 460 via the connector switch 430. The connector switch 430 may have two additional fluid connection points, one may be connected to a fresh dialysate source 440 (e.g., fresh PD dialysate bag) and the other end may be connected to a drain line or a spent dialysate container 442. In this setup, the PD effluent sampling system 400 may be connected to the PD tubing set 430 and/or the patient catheter 450 during any or all of the three phases of a PD treatment cycle.

The following describes the steps a user (e.g., a patient and/or caregiver) may perform as part of an exemplary method of performing a PET by utilizing the PD effluent sampling system 400, as illustrated in FIG. 4 in conjunction with a CAPD setup.

First, a patient may aseptically connect the tubing line 408 to the patient catheter 450 and the PD tubing set 460, for example via the connector switch 430 as shown in FIG. 4. Conversely, if the patient were not performing a PET, typically the patient would connect the patient catheter 450 to the PD tubing set 460 (e.g., via the connector 430), either directly or with a tubing extension.

The patient can open a clamp (not shown) on the patient line 450 to initiate draining of PD effluent to a spent dialysate container (e.g., 442). The patient may initiate a flush of the tubing. The patient may then switch to the fill phase, for example by rotating the connector switch 430 so the tube extension 408 is fluidly connected to the fresh dialysate source 440. Next, the patient may couple the PD effluent sampling system 400 to the tubing line 408, as shown in FIG. 4. Next, the patient may attach the PD effluent sampling system 400 to the tubing line 408. For example, this may include the durable components (e.g., sensor 420, clamp mechanisms 404/406, and controller 402). Attaching the PD effluent sampling system 400 can initiate a timer that marks the beginning of the dwell phase. The clamp mechanism 404 and the clamp mechanism 406 may start in a closed position. Next, the frangible (e.g., 412) may be broken (performed either by the PD effluent sampling system 400 or by the patient). The clamp 406 may be opened to enable PD effluent fluid to flow to the sampling container 410, passing the sensor 420, which may allow for calibration of the sensor 420 on fresh dialysate just introduced to the patient's peritoneal cavity. Because the frangible is now open, the fluid flows past the sensor 420, allowing calibration. Additionally, and/or alternatively, in some implementations, the PD effluent sampling system 400 may be couple to the tubing line 408 during the fill phase and the sensor 420 may be calibrated on fresh dialysate during the fill phase. If desired, the patient may disconnect from the PD tubing set 460.

Periodically during the dwell phase, PD effluent samples may be withdrawn and measured. The timing of these measurements maybe determined based on the timer. At the first measurement interval time mark, the PD effluent sampling system 400 may cause the tube clamp mechanism 406 to open for a specified amount of time, allowing a volume of PD effluent to flow from the patient's peritoneal cavity, past the sensor 420, and into the sample container 410. The tube clamp mechanism 406 can be closed to end the first sampling. In contrast to the PD effluent sampling system 300, the PD effluent sample may remain in the container 410, rather than being returned to the patient's peritoneal cavity. The step of opening the clamp 406 and taking measurements of a sample may be repeated at a second measurement interval time mark, a third measurement interval time mark, and additional measurement time marks, according to the instructions for the PET or the desired sampling schedule. The number of samples may be limited by the total volume of the sampling container 410.

At the end of the dwell phase, the patient may connect the tubing line 408 to a new PD tubing set 460 and may initiate a drain. The PD effluent sampling system 400 may be used to initiate a drain by opening the tube clamp mechanisms 404 and clamp 406. Subsequently, the PD effluent sampling system 400 may be disconnected from the tubing line 408. The PD effluent sampling system 400 may transmit/broadcast the sample measurements to a connected health device during or after the testing.

After the patient has drained, flushed the tubing, and filled the peritoneal cavity with fresh dialysate (if another exchange is to be performed), the patient (or a caregiver) may close a clamp on the patient line 450 and disconnect the tubing line 408.

In some implementations, the PD sampling system 400 may come as a kit and some of the components may be designed to be durable (reusable) while other components may be designed to be disposable. For example, one or more tubing lines 408 (with a frangible 412 and sample container 410) may come in a kit and these may be disposable components. The durable components of the kit may include a sensor 420, a controller 402, and tube clamp mechanisms 404/406.

In some implementations, the PD effluent sampling system 300/350/400 may operate with a series of manually operated mechanical clamps on the tubing extension 332 or the tubing line 408, and a patient or caregiver may manually open and close the clamps as directed, for example, from electronic prompts on a smartphone app that times the procedure.

In addition to a glucose sensor, the exemplary implementations described above may include other sensors, such as a turbidity sensor, which is useful in detecting peritonitis. Furthermore, PD effluent sampling systems may be utilized in a variety of sampling situations, where a sample drawn from a patient via a tube must be analyzed, regardless of connection to a larger, primary system.

In some instances, the exemplary implementations described above may be applied to a PD tubing set design for use with an APD cycler or for a PD tubing set designed for CAPD. In some implementations, the tubing section on which the PD effluent sampling system attaches (e.g., a tubing extension 332 or tubing line 408) may be integrated into a CAPD tubing set or APD tubing set or may exist as a standalone section to add when a membrane function assessment has been requested by the patient's care provider.

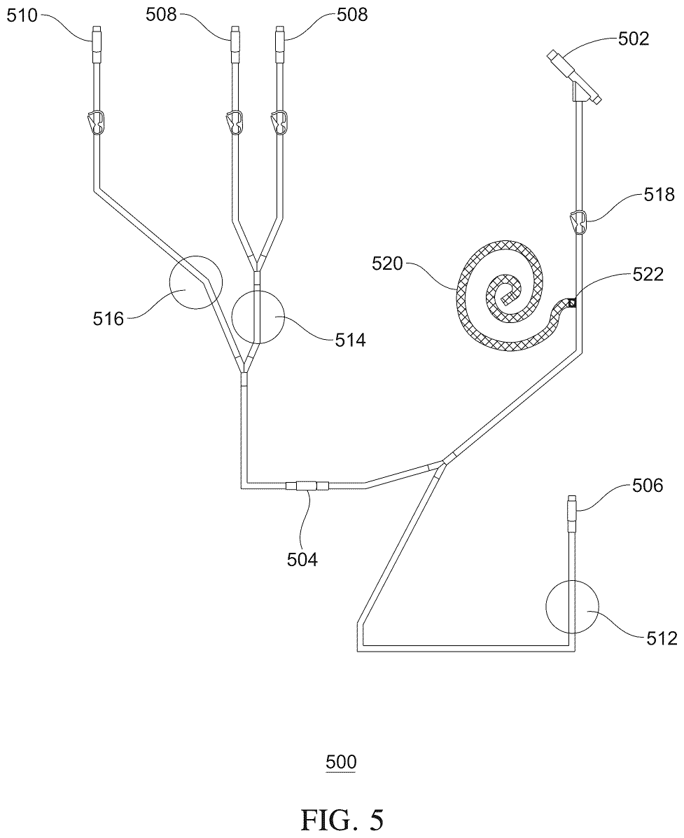

FIG. 5 is a schematic diagram depicting an exemplary implementation of how the disposable portion of a PD effluent sampling system, as described herein, may be integrated in or attachable to a PD tubing set. This PD tubing set example (which may be designed for use with a SILENCIA PD cycler offered by FRESENIUS MEDICAL CARE), has a coiled tubing line 520 illustrated as the representative sampling container that is integrated into the PD tubing set 500. However, it will be recognized that various types of sample containers can be utilized to store one or more samples withdrawn from the patient.

The PD tubing set 500 includes a patient connector 502 connectable to a patient line, a tube clamp mechanism 518 configured to control flow passing through the patient connector 502, and additional tubing lines connectable to a dialysate source(s) and a drain line or drain containers and corresponding tube clamp mechanisms. Although not shown in FIG. 5, the PD effluent sampling system (e.g., 300) may be attached to the PD tubing set 500 between the coiled tubing line 520 and the patient connector 502, in a similar arrangement as illustrated in FIG. 3A. This arrangement enables PD fluid that flows to the coiled tubing line 520 to pass by the one or more sensors of the respective PD effluent sampling system attached to the PD tubing set 500.

The PD tubing set 500 may offer enhanced flexibility by incorporating multiple tubing lines along with corresponding tube clamp mechanisms, as depicted in FIG. 5. For instance, a tubing line with a connector 506 may be connected to a drainage line or a drain bag to form a drain pathway. Optionally, a drain valve or drain clamp (not shown) may be disposed at position 512. Tubing lines extended to connectors 508 may be connected to fresh dialysate bags to supply fresh dialysate fluid. An inflow valve or clamp (not shown) may be disposed at position 514. Tubing lines extended to a connector 510 may enable a connection for an optional “last bag” (which may serve as a backup fresh dialysate source). An inflow valve or clamp (not shown) may be disposed at position 516. The PD tubing set 500 may further include a connector 504 that may be detachable. In some examples, after the treatment, the empty solution bags and corresponding tubing lines may be separated from the rest of the PD tubing set 500 by detaching the connector 504. Optionally, the empty solution bags may be used as drainage bags for the next treatment.

As detailed herein, PD effluent sampling systems provide two different approaches

to perform sampling and measurement. One approach allows PD effluent samples to be withdrawn and returned to the patient's peritoneal cavity, for example, as described in relation to the PD effluent sampling systems 300 and 350. This approach does not limit the number of samples by a total volume. The other approach involves withdrawing PD effluent samples without returning them into the patient's peritoneal cavity, for example, as described in relation to the PD effluent sampling system 400. To ensure that a minimum volume and/or concentration of PD effluent is retained within the patient's peritoneal cavity, the total sample volume may be constrained by a predetermined limit of the sample container.

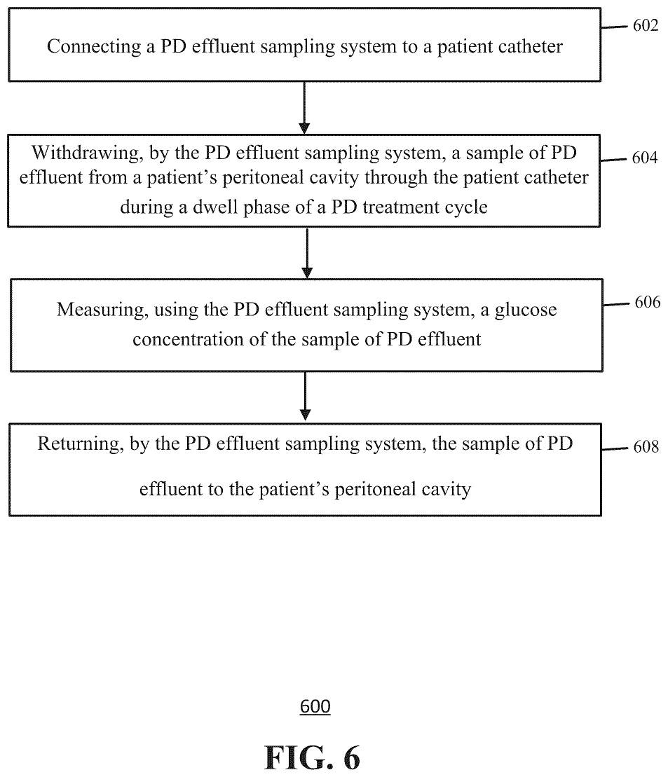

FIG. 6 is a flowchart of an exemplary process 600 for performing sample measurements using a PMFA effluent sampling system according to one or more examples of the present disclosure. The PD effluent sampling system may be any PD effluent sample system (e.g., 300, 350, 400) described in the present disclosure, or any combinations thereof. It will be recognized that any of the following blocks may be performed in any suitable order, and that the process 600 may be performed in any suitable environment. The descriptions, illustrations, and processes of FIG. 6 are merely exemplary, and the process 600 may use other descriptions, illustrations, and processes for sampling and collecting measurements.

As an example, the process 600 will be described in reference to the PD effluent sampling system 300 that includes a coiled tubing line (e.g., 310 in FIG. 3A) and a sensor device (e.g., the glucose sensor 320 as shown in FIG. 3A) configured to measure glucose concentrations. The coiled tubing line may have a first end and a second end, where the second end may be fluidly connectable to a patient catheter. The sensor device may be configured to measure a glucose concentration of PD effluent flowing to and/or from the coiled tubing line through the patient catheter.

At block 602, the PD effluent sampling system is connected to a patient catheter. For example, the second end of the coiled tubing line may be connected to the patient catheter via a T-connector. The PD effluent sampling system may be connected to the patient catheter before or during a dwell phase of a PD treatment cycle, which is not limited in the present disclosure.

At block 604, the PD effluent sampling system withdraws a sample of PD effluent from a patient's peritoneal cavity through the patient catheter during a dwell phase of a PD treatment cycle. For instance, the coiled tubing line may be uncoiled to cause PD effluent to flow into the coiled tubing line. In some examples, the coiled tubing line may be uncoiled by a motor (e.g., the motor 336 with the shaft 334 as depicted in FIG. 3A) based on instructions provided by a controller (e.g., the controller 330 as shown in FIG. 3A) in the PD effluent sampling system. In some instances, the coiled tubing line may be uncoiled based on user-input to a user-actuated handle attached to the first end of the coiled tubing line.

At block 606, the PD effluent sampling system measures the glucose concentration of the sample of PD effluent by using the sensor device. The sensor device may measure the glucose concentration as the PD effluent passes by the sensor. In some instances, the controller of the PD effluent sampling system may energize a motor to rotate a shaft (e.g., the shaft 334 as shown in FIG. 3A) to uncoil the coiled tubing line for a preset amount of time to obtain a sensor measurement from the sensor device. In some variations, the controller of the PD effluent sampling system may send alerts to prompt a user to uncoil the coiled tubing line for a preset amount of time to obtain a sensor measurement from the sensor device.

In further examples, the PD effluent sampling system may include additional sensors configured to measure temperature, conductivity, turbidity, or other suitable properties associated with the PD effluent sample and/or treatment process.

At block 608, the PD effluent sampling system returns the sample of PD effluent to the patient's peritoneal cavity. For instance, the coiled tubing line may be recoiled to cause the sample of PD effluent to flow back to the patient's peritoneal cavity. Similarly, the recoiling of the tubing line may be conducted manually or automatically. In one example, the controller of the PD effluent sampling system may energize the motor to rotate the shaft to cause the coiled tubing line to recoil. In another example, the controller of the PD effluent sampling system may send alerts to the user to prompt the user to uncoil the coiled tubing line.

The PD effluent sampling system may repeat blocks 604, 606, and 608 multiple times, so that the PD effluent sampling system may collect multiple sensor measurements for multiple samples of PD effluent during a dwell phase. For example, the PD effluent sampling system may be configured to repeat blocks 604, 606, and 608 at least three times to obtain three sets of measurements for three samples withdrawn from the patient's peritoneal cavity.

After measurements are completed, the PD effluent sampling system may be disconnected from the patient catheter. The PD effluent sampling system may transmit one or more measurements to a connected health device after each measurement, or after all measurements are completed, which is not limited in the present disclosure.

It will be recognized that the process 600 is applicable to other types of PD effluent sampling systems. For example, a PD effluent sampling system may include a fluid container having an expandable volume (such as the fluid container 352 as shown in FIG. 3B). With this type of PD effluent sampling system, PD effluent sample retrieval may be facilitated by expanding the expandable volume of the fluid container, while sample return may be facilitated by decreasing the expanded volume of the fluid container.

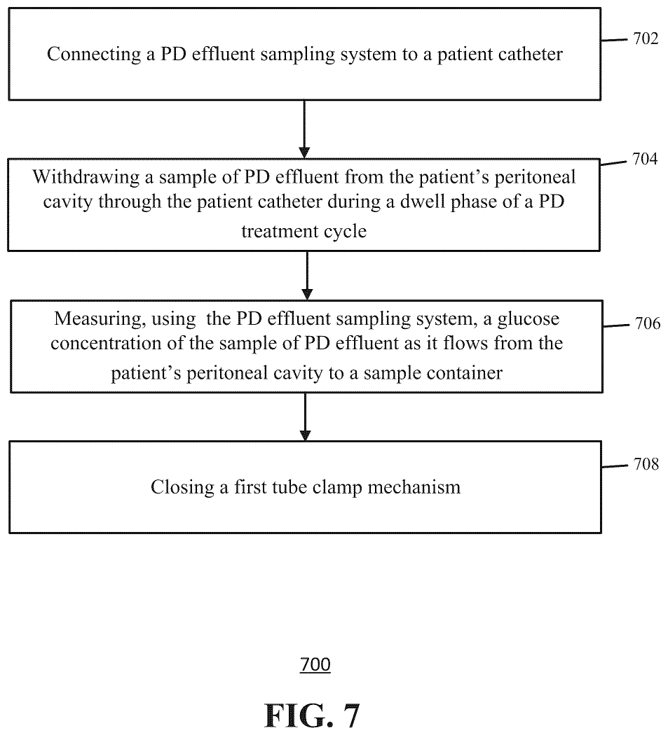

FIG. 7 is a flowchart of an exemplary process 700 for performing sample measurements using a PD effluent sampling system according to one or more examples of the present disclosure. The PD effluent sampling system may be any PD effluent sample system described in the present disclosure, or any combinations thereof. It will be recognized that any of the following blocks may be performed in any suitable order, and that the process 700 may be performed in any suitable environment. The descriptions, illustrations, and processes of FIG. 7 are merely exemplary, and the process 700 may use other descriptions, illustrations, and processes for sampling and collecting measurements.

As an example, the process 700 is demonstrated utilizing a PD effluent sampling system that includes a sample container (e.g., the sample container 410 as shown in FIG. 4), a sensor device (e.g., the sensor 420 as shown in FIG. 4) configured to measure glucose concentrations, a tubing line (e.g., the tubing line 408 as shown in FIG. 4) connected to a patient catheter, and one or more tube clamp mechanisms (e.g., the tube clamp mechanisms 404 and 406 as shown in FIG. 4). The sample container may be fluidly connected to the tubing line. The sensor device may be positioned along the tubing line and configured to measure a glucose concentration of PD effluent flowing from the patient catheter to the sample container. In some instances, the tubing line may stay connected to a PD tubing set (e.g., a APD or CAPD tubing set) when the PD effluent sampling system takes measurement.

At block 702, the PD effluent sampling system is connected to a patient catheter. For example, a tubing line (e.g., 408 as shown in FIG. 4) may be connected to a patient catheter and the PD effluent sampling system (e.g., 400) may be coupled to the tubing line. Before measurements, a first tube clamp mechanism (e.g., 406 as shown in FIG. 4) may be closed to block flow from the patient's peritoneal cavity to the sample container in the PD effluent sampling system. In some examples, a second tube clamp mechanism (e.g., 404 as shown in FIG. 4) may be closed to block flow from the sample container to a drain path. The tubing line and sample container may be fluidly connected, for example, by breaking the frangible between the two.

The PD effluent sampling system may be connected to the patient catheter before or during a dwell phase of a PD treatment cycle, which is not limited in the present disclosure.

At block 704, the PD effluent sampling system withdraws a sample of PD effluent from a patient's peritoneal cavity through the patient catheter during a dwell phase of a PD treatment cycle. For instance, the first tube clamp mechanism may be open for a set duration enabling PD effluent to flow from the patient's peritoneal cavity to the sample container through the tubing line. The first tube clamp mechanism may be operated automatically or manually. In one example, a controller (e.g., 402 as shown in FIG. 4) in the PD effluent sampling system may be configured to control individual clamp closures for the first tube clamp mechanism and/or the second tube clamp mechanism. In another example, the controller in the PD effluent sampling system may send alerts to prompt a user to close the first tube clamp mechanism and/or the second tube clamp mechanism.

At block 706, the PD effluent sampling system measures the glucose concentration of the sample of PD effluent by using the sensor device. The sensor device may measure the glucose concentration as the PD effluent passes by the sensor. In some instances, the controller of the PD effluent sampling system may open the first tube clamp mechanism for a preset amount of time to obtain a sensor measurement from the sensor device. In some variations, the controller of the PD effluent sampling system may send alerts to prompt a user to open the first tube clamp mechanism for a preset amount of time to obtain a sensor measurement from the sensor device.

In further examples, the PD effluent sampling system may include additional sensors configured to measure temperature, conductivity, turbidity, or other suitable properties associated with the PD effluent sample and/or treatment process.

At block 708, the PD effluent sampling system closes the first tube clamp mechanism. Similarly, individual clamp closures may be performed manually or automatically.

The PD effluent sampling system may repeat blocks 704, 706, and 708 multiple times, so that the PD effluent sampling system may collect multiple sensor measurements for multiple samples of PD effluent. For example, the PD effluent sampling system may be configured to repeat blocks 704, 706, and 708 three times to obtain three sets of measurements for three samples withdrawn from the patient.

After measurements are completed, the PD effluent sampling system (e.g., sensor 420, tube clamp mechanisms 404/406, and controller 402) may be disconnected from the tubing line. The PD effluent sampling system may transmit one or more measurements to a connected health device after each measurement, or after all measurements are completed, which is not limited in the present disclosure.

The systems and methods described in the present disclosure offer a number of advantages tailored to meet the diverse requirements of various medical settings and patients. For example, the systems and methods may be utilized with various PD types (e.g., APD and CAPD). These systems and methods can be integrated with existing PD assist devices (e.g., a SILENCIA cycler offered by FRESENIUS MEDICAL CARE), whether the PD devices use a pump or rely on gravity feed and/or drain. The systems and methods are also versatile in that they may be applied in home, clinical, or critical care environments. The systems and methods may be integrated into a digital PD platform. Lastly, the systems and method can simplify reinfusion, eliminating the inconvenience of losing PD effluent (e.g., 200 ml at a time) during a membrane function assessment, a drawback often associated with other methods that can affect the efficacy of dialysis therapy.

It will be appreciated that the various machine-implemented operations described herein may occur via the execution, by one or more respective processors, of processor-executable instructions stored on a tangible, non-transitory computer-readable medium, such as a random access memory (RAM), read-only memory (ROM), programmable read-only memory (PROM), and/or another electronic memory mechanism. Thus, for example, operations performed by any device described herein may be carried out according to instructions stored on and/or applications installed on the device, and via software and/or hardware of the device.

All references, including publications, patent applications, and patents, cited herein are hereby incorporated by reference to the same extent as if each reference were individually and specifically indicated to be incorporated by reference and were set forth in its entirety herein.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present disclosure covers further embodiments with any combination of features from different embodiments described above and below.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A method of sampling of peritoneal dialysis (PD) effluent for a peritoneal membrane function assessment, comprising:
(a) fluidly connecting a PD effluent sampling system to a patient catheter, wherein the PD effluent sampling system comprises:
a coiled tubing line having a first end and a second end fluidly connectable to the patient catheter; and
a sensor device configured to measure a glucose concentration of PD effluent flowing to and/or from the coiled tubing line through the patient catheter;
(b) withdrawing, by uncoiling the coiled tubing line, a sample of PD effluent from a patient's peritoneal cavity through the patient catheter during a dwell phase of a PD treatment cycle;
(c) measuring, using the sensor device, the glucose concentration of the sample of PD effluent; and
(d) returning, by recoiling the coiled tubing line, the sample of PD effluent to the patient's peritoneal cavity.

2. The method according to claim 1, further comprising:
performing steps (b)-(d) at least three times periodically during the dwell phase.

3. The method according to claim 1, wherein the PD effluent sampling system further comprises:
a controller;
a motor connectable to the first end of the coiled tubing line; and
a power source operably connected to the controller and the motor,
wherein the controller is configured to provide control instructions to the motor to coil or uncoil the coiled tubing line.

4. The method according to claim 3, wherein the PD effluent sampling system further comprises:
a shaft having a first end and a second end, wherein the first end of the shaft is connected to the motor, and wherein the second end of the shaft is attachable to the first end of the coiled tubing line,
wherein the controller is further configured to provide control instructions to the motor to actuate the shaft to coil or uncoil the coiled tubing line.

5. The method according to claim 4, wherein the second end of the shaft is coupled to a tubing connector, and wherein the first end of the coiled tubing line is attachable to the second end of the shaft through the tubing connector.

6. The method according to claim 4, wherein the controller is further configured to:
provide first control instructions to actuate the shaft to uncoil the coiled tubing line for a first amount of time to obtain a first sensor measurement from the sensor device; and
provide second control instructions to actuate the shaft to recoil the coiled tubing line. 7 The method according to claim 6, wherein the controller is further configured to:
provide the first control instructions to actuate the shaft to uncoil the coiled tubing line for the first amount of time to obtain a second sensor measurement from the sensor device; and
provide the second control instructions to actuate the shaft to recoil the coiled tubing line.

8. The method according to claim 7, wherein the controller is further configured to:
provide the first control instructions to actuate the shaft to uncoil the coiled tubing line for the first amount of time to obtain a third sensor measurement from the sensor device; and
provide the second control instructions to actuate the shaft to recoil the coiled tubing line.

9. The method according to claim 8, wherein the PD effluent sampling system further comprises a transmitter, and wherein the method further comprises:
disconnecting the PD effluent sampling system from the patient catheter; and
transmitting, by the PD effluent sampling system through the transmitter, the first, second, and third measurements to a connected health device.

10. The method according to claim 1, further comprising:
measuring, using the sensor device, other properties of the sample of PD effluent, wherein the other properties comprises at least one of:
temperature;
conductivity; and
turbidity.

11. The method according to claim 1, wherein the sensor device further comprises a turbidity sensor, and the turbidity sensor is configured to detect peritonitis based on the sample of PD effluent.

12. A method of sampling of peritoneal dialysis (PD) effluent for a peritoneal membrane function assessment, comprising:
(a) fluidly connecting a PD effluent sampling system to a patient catheter, wherein the PD effluent sampling system comprises:
a fluid container having an expandable volume fluidly connectable to the patient catheter; and
a sensor device configured to measure a glucose concentration of PD effluent flowing to and/or from the fluid container through the patient catheter;
(b) withdrawing, by expanding the expandable volume of the fluid container, a sample of PD effluent from a patient's peritoneal cavity through the patient catheter during a dwell phase of a PD treatment cycle;
(c) measuring, using the sensor device, the glucose concentration of the sample of PD effluent; and
(d) returning, by decreasing the expanded volume of the fluid container, the sample of PD effluent to the patient's peritoneal cavity.

13. A peritoneal dialysis (PD) effluent sampling system, comprising:
a coiled tubing line having a first end and a second end fluidly connectable to a patient catheter; and
a sensor device configured to measure a glucose concentration of PD effluent flowing to and/or from the coiled tubing line through the patient catheter;
wherein:
the coiled tubing line is configured to uncoil to withdraw a sample of PD effluent from a patient's peritoneal cavity through the patient catheter during a dwell phase of a PD treatment cycle;
the sensor device is configured to measure the glucose concentration of the sample of PD effluent; and
the coiled tubing line is configured to recoil to return the sample of PD effluent to the patient's peritoneal cavity.

14. The system according to claim 13, wherein the coiled tubing further comprises a user-actuated handle that is attached to the first end of the coiled tubing line, wherein the user-actuated handle, based on user-input, uncoils and recoils the coiled tubing line.

15. The system according to claim 13, wherein the PD effluent sampling system further comprises:
a controller;
a motor connectable to the first end of the coiled tubing line; and
a power source operably connected to the controller and the motor,
wherein the controller is configured to provide control instructions to the motor to coil or uncoil the coiled tubing line.

16. The system according to claim 15, wherein the PD effluent sampling system further comprises:
a shaft having a first end and a second end, wherein the first end of the shaft is connected to the motor, and wherein the second end of the shaft is attachable to the first end of the coiled tubing line,
wherein the controller is further configured to provide control instructions to the motor to actuate the shaft to coil or uncoil the coiled tubing line.

17. The system according to claim 16, wherein the controller is further configured to periodically withdraw samples of PD effluent from the patient's peritoneal cavity during the dwell phase of the PD treatment cycle by:
providing, for withdrawing each sample of the samples of PD effluent, first control instructions to actuate the shaft to uncoil the coiled tubing line for the first amount of time to withdraw the particular sample of the samples of PD effluent from the patient's peritoneal cavity;
obtaining, from the sensor device, a glucose concentration measurement of the particular sample of PD effluent; and
providing second control instructions to actuate the shaft to recoil the coiled tubing line to return the particular sample of the samples of PD effluent to the patient's peritoneal cavity.

18. The system according to claim 17, further comprising:
a transmitter configured to transmit the glucose concentration measurements of the samples of PD effluent to a connected health device.

19. The system according to claim 13, wherein the sensor device is further configured to measure at least one of:
temperature;
conductivity; and
turbidity.

20. The system according to claim 13, wherein the sensor device further comprises a turbidity sensor, and the turbidity sensor is configured to detect peritonitis based on the sample of PD effluent.
